(19) United States
(12) Patent Application Publication
(10) Pub. No.: US 2009/0266182 A1
(43) Pub. Date: Oct. 29, 2009

(54) METHOD AND INSPECTING DEVICE FOR IDENTIFYING TEST SPECIMEN AND MULTIFUNCTIONAL TEST SPECIMEN

(75) Inventor: Meng-Yi Lin, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: HEALTH & LIFE CO., LTD., Taipei (TW)

(21) Appl. No.: 12/410,065

(22) Filed: Mar. 24, 2009

(30) Foreign Application Priority Data
Mar. 24, 2008 (TW) 097110417

(51) Int. Cl. G01N 33/00; G01N 27/00

(52) U.S. Cl. 73/865.8; 324/71.1

(57) ABSTRACT

An inspecting device with an identification function is provided to identify and analyze different types of test specimens. (The test specimens include information to be measured and/or identification information.) In order to enable the inspecting device to identify the test specimen, an appropriate electrode pattern corresponding to an electrode in a specimen connection port of the inspecting device is arranged on one end of the test specimen. During a process of inserting the test specimen from an open end of the specimen connection port to reach a bottom end thereof, a logic change based on the electrode pattern detected by the connection port is utilized to identify the type of the specimen or read the information to be measured. A method for identifying different types of test specimens and a multifunctional test specimen is also provided.

### FIELD OF THE INVENTION

The present invention relates to an inspecting device with an identification function, in particular an in vitro diagnostic (IVD) device which is capable of identifying a plurality of analysis specimens.

### DESCRIPTION OF THE PRIOR ART

The IVD device refers to any reagent, calibration substance, control substance, instrument, device, equipment, system, or any other medical instrument used to inspect samples (including blood and tissue) collected from a human body in vitro, which is used individually or mainly to determine the health status, thereby curing, relieving, remedying, or guarding against diseases or sequelae. Most IVD devices need to be used together with specific specimens, but each batch of test specimens varies in quality, so the inspecting device needs to obtain the calibration data of the specimen before performing the measurement.

The IVD device may need to identify different specimens, and then perform corresponding functions accordingly, for example, an inspecting instrument may be used together with different test specimens and capable of inspecting different inspecting targets, or an inspecting instrument may be capable of identifying the control information of different batches of specimens. Accordingly, such design involves the problem of identification of a plurality of test specimens by a single instrument or device.

In prior art, two kinds of designs can be used to achieve the above identification. In the first kind of design, an inspecting instrument using a calibration code card stores identification information of the test specimen in the calibration code card, and the inspecting instrument first reads the data stored in the calibration code card before the specimen is inserted for measurement. At this time, the instrument can confirm the type of the test specimen and thus perform the corresponding procedure. In the other kind of design, an electrode is arranged on the test specimen, and after the test specimen is inserted into the inspecting instrument, the type of the test specimen is determined according to the logic state of an electrode pin on a connection port of the instrument, thereby achieving the identification.

The first kind of design can only be used in structures configured with the calibration code card, and the manufacture cost thereof is relatively high. In the second kind of design, the number of the logic states that can be determined is limited by the number of pins on the connection port of the instrument and the area of the specimen. (The configuration of the electrode on the test specimen and the logic states determined by the electrode pins on the connection port of the instrument are fixed, and the area of the electrode required is relatively large while the number thereof is limited.)

### SUMMARY OF THE INVENTION

In order to solve the problems in prior art, the present invention provides an inspecting device with a test specimen identification function, an identifying method, and a test specimen used together with the inspecting device, so that the same device can identify different test specimens.

Through an electrode distribution on the test specimen, when the test specimen is inserted into an inspecting device including a connection port, the electrodes of the test specimen will generate and output signals when sensing an electrode in the connection port in a contacting or non-contacting manner. Through the electrode distribution on the test specimen, during the process of inserting the test specimen from an open end of the connection port to reach a bottom end thereof, a logic state change of an indicator distribution pattern detected by the connection port is utilized to determine the type of the test specimen. Therefore, the electrode pins on the connection port of the inspecting device and the indication electrodes on the test specimen are greatly simplified according to the present invention.

The present invention provides a multifunctional inspecting device, which includes one or more test specimen connection ports and one or more test specimens. Each test specimen connection port has an open end and a bottom end. Each test specimen includes: a substrate; a plurality of strip-shaped regions, located on at least one surface of the substrate, each strip-shaped region including one or more strip-shaped indicators; and at least one reaction region, located on at least one surface of the substrate and used to accept and analyze one or more samples. An indicator distribution pattern is formed on the substrate by using the existence, nonexistence or amount of the strip-shaped indicators on each of the plurality of strip-shaped regions, in which the indicator distribution pattern is assigned to indicate one or more identification types.

The present invention further provides a method for identifying a test specimen, which includes the following steps. A plurality of strip-shaped regions including or not including a plurality of strip-shaped regions with different lengths is formed on a test specimen. A plurality of receptors corresponding to the plurality of different strip-shaped indicators is formed in a test specimen connection port. The test specimen is inserted from an open end of the test specimen connection port to a bottom end thereof. The plurality of receptors detects the existence, nonexistence, or change of the strip-shaped indicators during a process of inserting the test specimen from the open end of the test specimen connection port to the bottom end thereof. The detected existence, nonexistence, or change of the strip-shaped indicators is output as a logic change mode. The logic change mode is assigned as the test specimen's identification information or information to be measured.

The present invention further provides a test specimen, which is used together with an inspecting device having a connection port and having a plurality of measuring functions. The test specimen includes: a substrate; a plurality of strip-shaped regions, located on at least one surface of the substrate, each strip-shaped region including one or more strip-shaped indicators; and at least one reaction region, located on at least one surface of the circuit substrate and used to accept and analyze one or more samples, in which an indicator distribution pattern is formed on the substrate by using the existence, nonexistence or amount of the strip-shaped indicators on each of the plurality of strip-shaped regions, in which the indicator distribution pattern is assigned to indicate one or more identification types.

To make other features and advantages of the present invention more comprehensible, the present invention is further demonstrated below in detail with reference to the accompanying drawings, detailed descriptions, and preferred embodiments.

### BRIEF DESCRIPTION OF THE DRAWINGS

FIGS. 1(a), (b), and (c) are top views of (a part of) an inspecting device for identifying specimens, and a process of inserting a test specimen to be identified according to a preferred embodiment of the present invention;

FIGS. 2(a) to (d) show the inspecting device and the test specimen according to the preferred embodiment of the present invention;

FIG. 3 is a schematic view of an electrode circuit at a test specimen connection port in the inspecting device according to the preferred embodiment of the present invention;

FIG. 4(a) shows square waveforms transferred to the inspecting device through an electrode P-A and an electrode P-B of the connection port during the process of inserting the test specimen to be inspected; and FIG. 4(b) shows identification value sequences read after the electrode P-A and the electrode P-B of the connection port react with test specimen indication electrodes during the process of inserting the test specimen to be inspected;

FIG. 5(a) shows four different examples of the changing aspects of a distribution pattern of specimen indication electrodes corresponding to the connection port P-B of the inspecting device shown in FIG. 3; and FIG. 5(b) shows four identification value sequences read by the connection port P-B of the inspecting device during the process of inserting the four test specimens shown in FIG. 5(a);

FIG. 6(a) shows distribution patterns of four test specimen indication electrodes and the correspondingly assigned specimen types according to the preferred embodiment of the present invention; and FIG. 6(b) shows four identification value sequences read by the connection port P-B of the inspecting device during the process of inserting the four test specimens shown in FIG. 6(a);

FIG. 7 shows distribution patterns of 15 test specimen indication electrodes and the correspondingly assigned specimen types according to a preferred embodiment of the present invention; and

FIG. 8 shows distribution patterns of 15 test specimen indication electrodes and the correspondingly assigned specimen types according to a preferred embodiment of the present invention.

### DETAILED DESCRIPTION

It should be noted that, although an exemplary specific embodiment is taken as an example in the entire discussion, other alternative embodiments may also include various aspects and fall within the scope of the present invention.

In order to enable an inspecting device to identify a test specimen, an appropriate electrode pattern corresponding to a specimen connection port of the inspecting device is arranged on one end of the test specimen. During a process of inserting the test specimen from an open end of the specimen connection port to reach a bottom end of the connection port, a logic state change of the electrode pattern detected by the connection port is assigned as specific identification information of the test specimen, and then the test specimen is identified by the inspecting device.

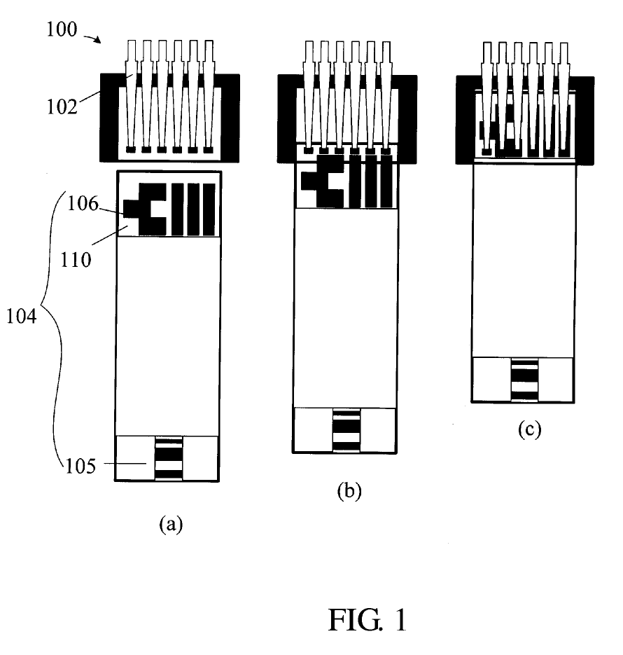

FIGS. 1(a), (b), and (c) are top views of (a part of) an inspecting device for identifying specimens, and a process of inserting a test specimen to be identified according to a preferred embodiment of the present invention. An electrode 102 of a test specimen connection port 100 in the inspecting device contacts an indication electrode 106 of a test specimen 104, and an analyzing signal of a reaction region 105 for accepting and analyzing one or more samples in the test specimen 104 is electrically transmitted to the inspecting device (not shown) through the electrode 102 of the connection port.

The test specimen 104 to be identified may be repeatedly inserted into and pulled from the test specimen connection port 100. When the test specimen 104 is inserted into the connection port 100 during the processes of FIGS. 1(a), (b), and (c), each single electrode in the electrode 102 of the connection port corresponds to each indication electrode 106 on the test specimen 104. On the basis of the electrode distribution pattern of each indication electrode in an inserting and/or pulling direction, a specific logic signal is generated due to the change of the relative position between each indication electrode and the electrode of the connection port during the inserting and/or pulling process. Therefore, the electrode 102 of the connection port transmits the logic signal generated due to the change of the relative position between the indication electrode 106 in a jointing region 110 and the electrode 102 of the connection port during the process of inserting or pulling the test specimen 104 to the inspecting device for identification and determination.

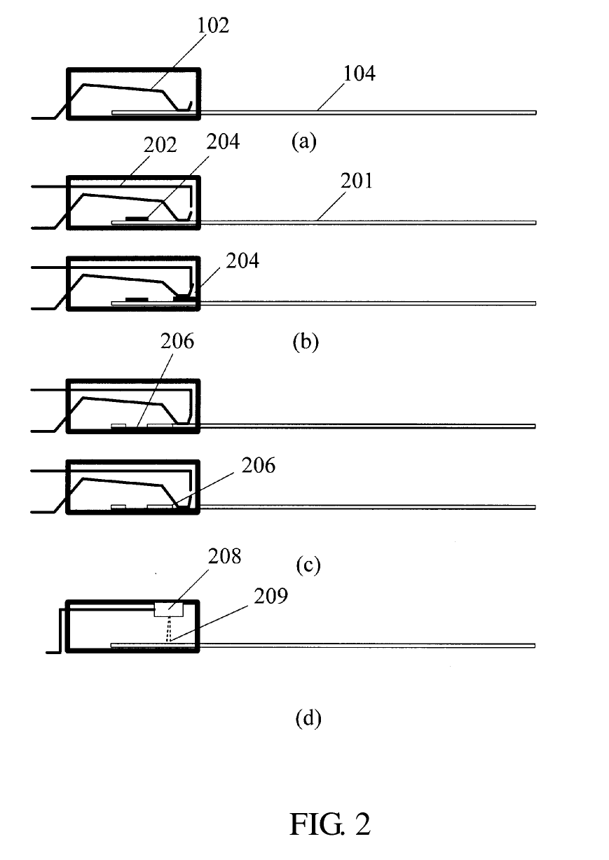

FIGS. 2(a) to (d) show the inspecting device and the test specimen according to the preferred embodiment of the present invention. In different implementation aspects, the existence or nonexistence of the electrodes on the test specimen may be replaced by an existence or nonexistence of protrusions/recesses in a mechanical component or an optical sensing element.

FIG. 2(a) is a side view of the electrode 102 of the connection port, the test specimen 104, and the indication electrode 106 of FIG. 1. Through the change in the design of the mechanical component, the protrusion 204 on the test specimen 201 of FIG. 2(b) and the recess 206 of FIG. 2(c) are configured appropriately, so that an electrode 202 of the connection port of the inspecting device generates a corresponding circuit conduction effect ((1) to (2) of FIG. 2(b)) when encountering the protrusion or generates a corresponding circuit break effect ((1) to (2) of FIG. 2(c)) when encountering the recess, thereby identifying the test specimen. FIG. 2(d) shows that an optical inspecting port 208 on the inspecting device senses the existence, nonexistence, or distribution of an optical material 209 capable of being sensed by the optical inspecting port 208 on the test specimen to be inspected, thereby determining and identifying the type of the test specimen.

Therefore, by using the electrode detection between the inspecting device and the test specimen to be inspected or the conducting or breaking effect of the circuit produced on the inspecting device due to the design of the mechanical components, or by using the optical sensing elements for replacement, the logic signal capable of identifying the type or the inspecting data of the test specimen to be inspected is generated, and the signal is transmitted by the connection port of the inspecting device or the electrode of the connection port.

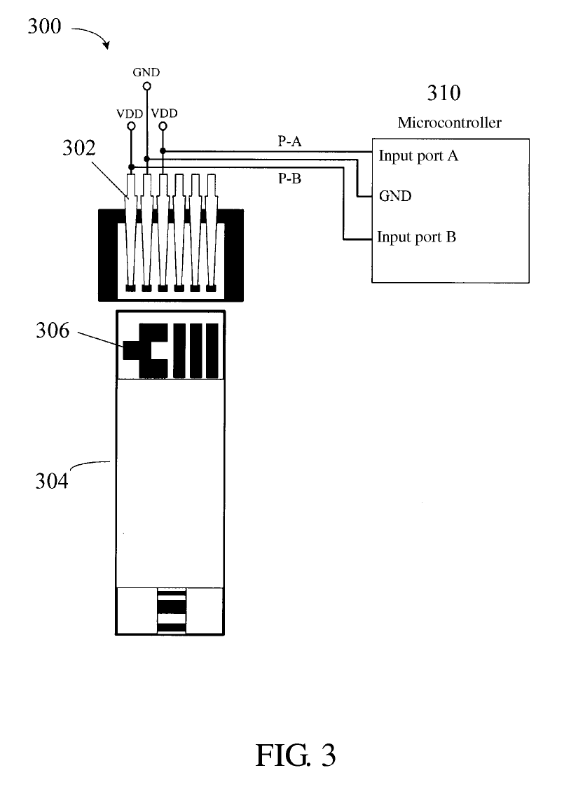

FIG. 3 is a schematic view of an electrode circuit at a test specimen connection port in the inspecting device according to the preferred embodiment of the present invention. An electrode P-A and an electrode P-B of the connection port are connected to a microcontroller 310 in the inspecting device, and GND represents a grounding electrode. The electrode P-A and the electrode P-B of the connection port correspond to the electrodes on the test specimen.

When the test specimen 304 is inserted into the connection port, through a simple design of connecting to the microcontroller 310, the received analogue identification signal is converted into a digital output of pulse timing waveforms, which is helpful for the subsequent identification of the type of the test specimen.

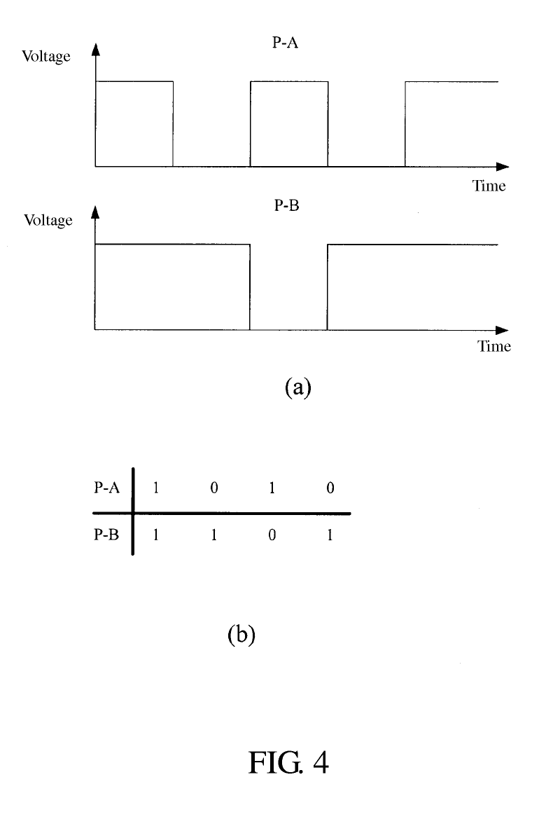

FIG. 4(a) shows pulse waveforms transmitted to the inspecting device through an electrode P-A and an electrode P-B of the connection port during the process of inserting the test specimen to be inspected as shown in FIG. 3. For example, as for the electrode P-A of the connection port of FIG. 3, the strip-shaped indication electrodes on the corresponding test specimen along the inserting direction may be divided into three square regions, in which black represents existence of electrodes in the square region, and white represents nonexistence. Once the test specimen is inserted, the electrode P-A of the connection port encounters the part of the indication electrode region having electrodes on the corresponding test specimen to generate the conducting effect, and in this case, a square wave intensity is 0, and the identification value is 0. When the electrode P-A of the connection port encounters the part without electrodes to generate the breaking effect, the pulse intensity is 1, and the identification value is set to 1. As for the electrode P-B, only the second part of the corresponding indication electrode has electrodes, so the time square wave intensity corresponding to the second part is 0 only when the test specimen is inserted, that is, the identification value is 0.

FIG. 4(b) shows identification value sequences read after the electrode P-A and the electrode P-B of the connection port react with test specimen indication electrodes during the process of inserting the test specimen to be inspected, in which P-A is 1010 and P-B is 1101.

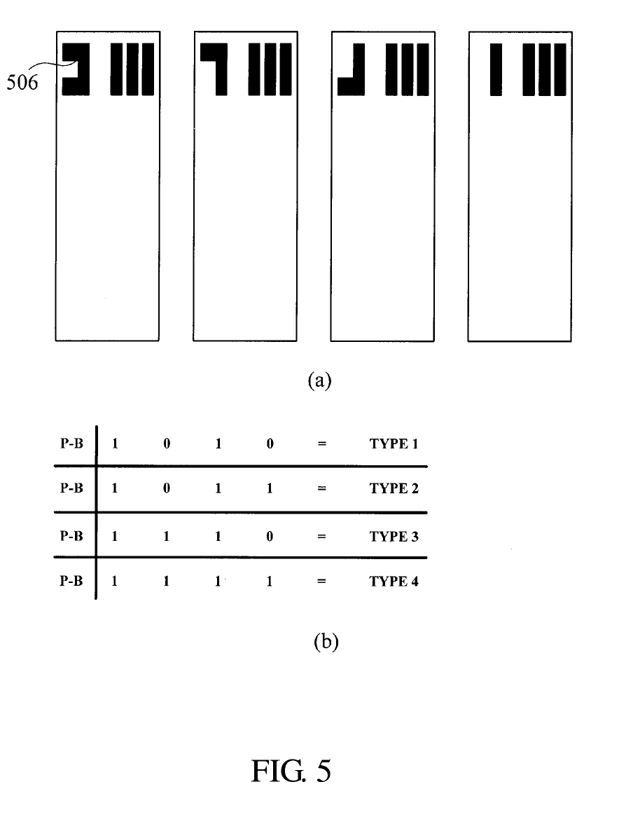

FIG. 5(a) shows four different examples of the changing aspects of a distribution pattern of specimen indication electrodes corresponding to the connection port P-B of the inspecting device shown in FIG. 3. Similarly, the identification value of the part of the indication electrode region having electrodes on the test specimen is read as 0; otherwise, it is 1.

The indication electrodes in the test specimen of the present invention are configured as strip-shaped, and the quantity of the indication electrodes may be determined according to the actual demands. In this embodiment (FIG. 3), the region is further divided into three square regions by three strip-shaped indication electrodes, and electrodes are placed or not placed in the three regions. During the process of inserting the test specimen into the test specimen connection port along a single direction, each strip-shaped indication electrode of the test specimen contacts with the receiving electrode on the connection port to form a closed loop, thereby producing a current. Then, by using the respective specific digital output of pulse timing waveforms (as shown in FIG. 4(b)) of the voltage with the time elapsed during the inserting process, the logic change mode is set and the identification value sequence is set, thereby identifying the pre-assigned type of the test specimen.

FIG. 5(b) shows four identification value sequences read by the connection port P-B of the inspecting device during the process of inserting the four test specimens shown in FIG. 5(a), and the four identification value sequences are set to TYPE 1, TYPE 2, TYPE 3, and TYPE 4.

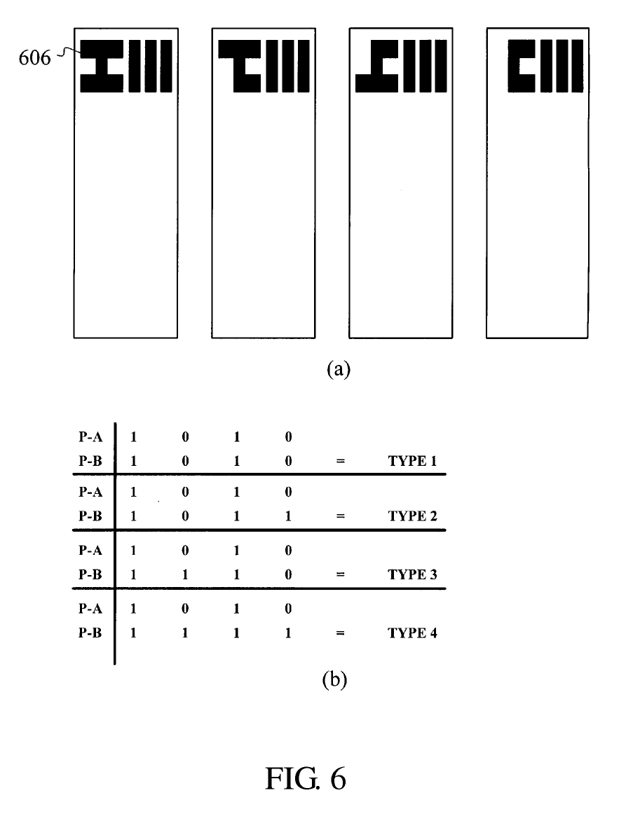

FIG. 6(a) shows distribution patterns of four test specimen indication electrodes and the correspondingly assigned specimen types according to the preferred embodiment of the present invention. During the process of inserting the test specimen into the connection port, the part of the test specimen having indication electrodes is assigned to logic 1, and the part without indication electrodes is 0. According to a combination of 1 and 0 (a combination of the three electrode sequences in this embodiment), TYPE 1, TYPE 2, TYPE 3, and TYPE 4 of the test specimen as shown in FIG. 6(b) are assigned. In this embodiment, the electrode P-A of the connection port corresponding to that shown in FIG. 3 is taken as a reference for the detection of the repeated inserting and pulling motions, and only the identification value of the P-B is used to set the identification type of the specimen.

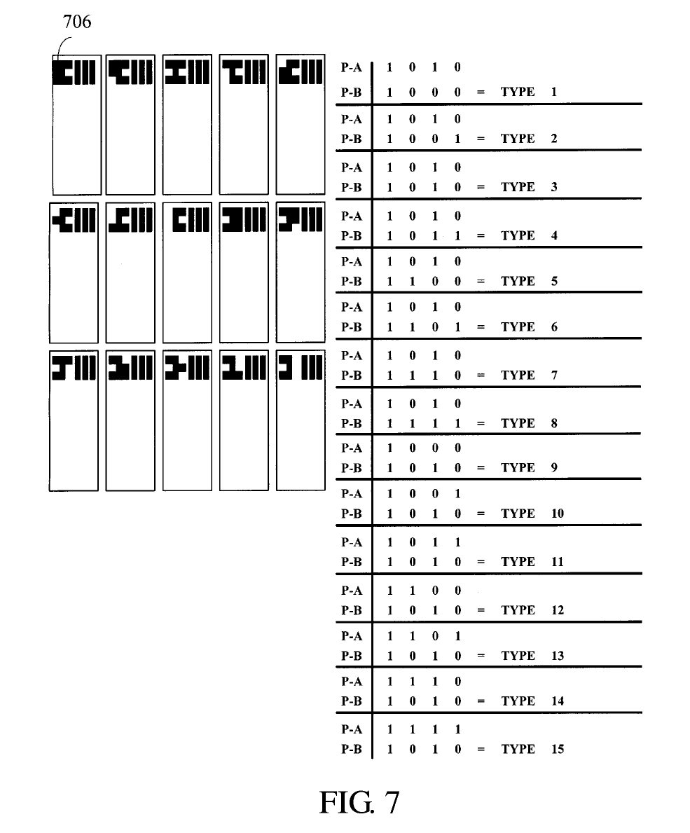

FIG. 7 shows distribution patterns of 15 test specimen indication electrodes and the correspondingly assigned specimen types according to a preferred embodiment of the present invention. In this embodiment, the electrodes P-A and P-B of the connection port corresponding to that shown in FIG. 3 are alternately used as the reference, and are alternately used for identification, so as to set the identification type (TYPE1 to TYPE15) of the specimen. For the 1^{st} to 8^{th} types, the P-A is used as the reference, and for the 9^{th} to 15^{th} types, the P-B is used as the reference; when used as the reference, the electrodes P-A and P-B have the same electrode pattern on the respective corresponding test specimen.

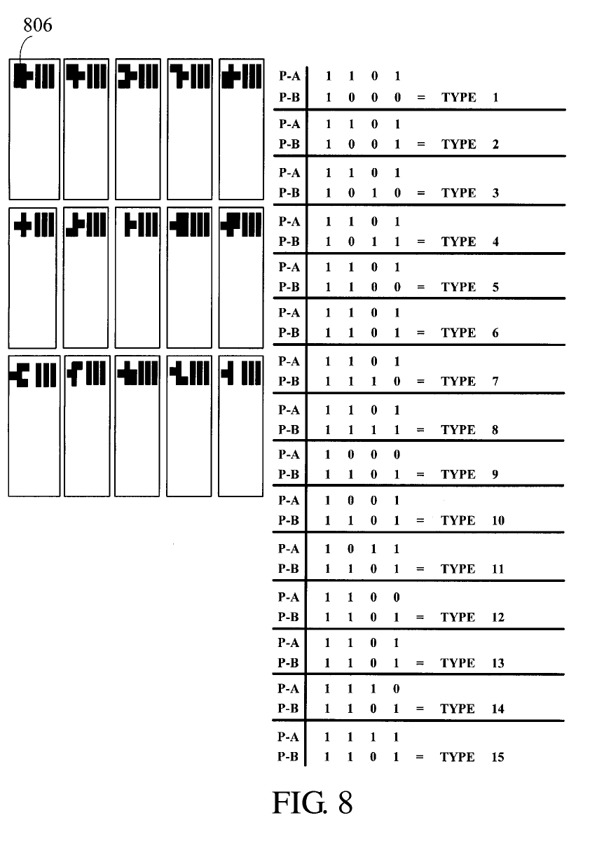

FIG. 8 shows distribution patterns of 15 test specimen indication electrodes and the correspondingly assigned specimen types according to a preferred embodiment of the present invention. In this embodiment, the electrodes P-A and P-B of the connection port corresponding to that shown in FIG. 3 are alternately used as the reference, and are alternately used for identification, so as to set the identification type (TYPE1 to TYPE15) of the specimen. For the 1^{st} to 8^{th} types, the P-A is used as the reference, and for the 9^{th} to 15^{th} types, the P-B is used as the reference; when used as the reference, the electrodes P-A and P-B have opposite electrode patterns on the respective corresponding test specimen.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents. For example, although in the preferred embodiment, at most 6 regions are provided for placing the strip-shaped indicators (for example, electrodes or optical sensing elements) of the test specimen, those skilled in the art will know that decreasing or increasing the number of the indicator regions falls within the scope of the present invention.

We claim:

1. An inspecting device with an identification function, comprising:
one or more test specimen connection ports, having an open end and a bottom end; and
one or more test specimens, wherein each of the test specimens comprises:
a substrate;
a plurality of strip-shaped regions, located on at least one surface of the substrate, each strip-shaped region comprising one or more strip-shaped indicators; and
at least one reaction region, located on at least one surface of the substrate and used to accept and analyze one or more samples, wherein an indicator distribution pattern is formed on the substrate by using the existence, nonexistence or amount of the strip-shaped indicators on each of the plurality of strip-shaped regions, and a logic state change mode represented by the indicator distribution pattern is assigned as identification information of the test specimen.

2. The inspecting device according to claim 1, wherein when the logic state change mode represented by the indicator distribution pattern is assigned as the identification information of the test specimen, the logic change of the indicator distribution pattern detected by a plurality of receptors in the test specimen connection port during a process of inserting the test specimen from the open end of the test specimen connection port to reach the bottom end thereof is assigned as the identification information of the test specimen.

3. The inspecting device according to claim 1 or 2, wherein the plurality of receptors in the one or more test specimen connection ports corresponds to the plurality of strip-shaped regions in the one or more test specimens one by one.

4. The inspecting device according to claim 1 or 2, wherein the plurality of strip-shaped regions and the at least one reaction region are located on the same or different surfaces of the substrate.

5. The inspecting device according to claim 2, wherein the one or more strip-shaped indicators are electrical materials, optical sensing materials, or mechanical structures.

6. The inspecting device according to claim 2, wherein the indicator distribution pattern is detected in an electrical contacting, optical sensing, or mechanical contacting manner.

7. The inspecting device according to claim 3, wherein the plurality of receptors is electrically conductive elements, optical sensing elements, or mechanical contacting structures.

8. An identifying method for a test specimen, comprising:
forming a plurality of strip-shaped regions on a test specimen, wherein the plurality of strip-shaped regions comprises or does not comprise a plurality of strip-shaped indicators of different lengths;
forming a plurality of receptors in a test specimen connection port, wherein the plurality of receptors corresponds to the plurality of strip-shaped indicators;
inserting the test specimen from an open end of the test specimen connection port to a bottom end thereof;
detecting the existence, nonexistence, or change of the strip-shaped indicators by the plurality of receptors during a process of inserting the test specimen from the open end of the test specimen connection port to the bottom end thereof;
outputting the detected existence, nonexistence, or change of the strip-shaped indicators as a logic state change; and
assigning the logic state change as identification information of the test specimen.

9. The identifying method according to claim 8, wherein the plurality of strip-shaped indicators is electrical materials, optical sensing materials, or mechanical structures.

10. The identifying method according to claim 8, wherein the plurality of receptors detects by means of electrical contacting, optical sensing, or mechanical contacting.

11. The identifying method according to any one of claims 8 to 10, wherein the plurality of receptors in the test specimen connection port corresponds to the plurality of strip-shaped regions in the test specimen one by one.

12. The identifying method according to any one of claims 8 to 10, wherein the plurality of receptors is electrically conductive elements, optical sensing elements, or mechanical contacting structures.

13. A multifunctional inspecting device, which has connection ports and suitable for use with a test specimen with multiple test functions, the test specimen comprising:
a circuit substrate;
a plurality of strip-shaped regions, located on at least one surface of the circuit substrate, each strip-shaped region comprising one or more strip-shaped indicators; and
at least one reaction region, located on at least one surface of the circuit substrate and used to accept and analyze one or more samples,
wherein an indicator distribution pattern is formed on the circuit substrate by using the existence, nonexistence or amount of the strip-shaped indicators on each of the plurality of strip-shaped regions, wherein the indicator distribution pattern is assigned as identification information of the test specimen.

14. The multifunctional inspecting device according to claim 13, wherein when the indicator distribution pattern is assigned as a type of the one or more samples, a logic state change of the indicator distribution pattern detected by a test specimen connection port during a process of inserting the test specimen from an open end of the test specimen connection port to a bottom end of the connection port is assigned as the identification information of the test specimen.

15. The multifunctional inspecting device according to claim 13 or 14, wherein the one or more test specimen connection ports comprise a plurality of receptors corresponding to the plurality of strip-shaped regions in the one or more test specimens one by one.

16. The multifunctional inspecting device according to claim 13 or 14, wherein the plurality of strip-shaped regions and the at least one reaction region are located on the same or different surfaces of the circuit substrate.

17. The multifunctional inspecting device according to claim 13, wherein the one or more strip-shaped indicators are electrical materials, optical sensing materials, or mechanical structures.

18. The multifunctional inspecting device according to claim 14, wherein the indicator distribution pattern is detected by means of electrical contacting, optical sensing, or mechanical contacting.

19. The multifunctional inspecting device according to claim 15, wherein the plurality of receptors is electrically conductive elements, optical sensing elements, or mechanical contacting structures.
